(19) United States
(12) Patent Application Publication
(10) Pub. No.: US 2021/0298961 A1
(43) Pub. Date: Sep. 30, 2021

(54) BIODEGRADABLE, COMPOSTABLE DIAPER AND METHOD OF MANUFACTURE THEREOF

(71) Applicant: ZETA GROUP (NZ) LIMITED, Auckland (NZ)

(72) Inventor: Sheng LIU, Wellington (NZ)

(73) Assignee: ZETA GROUP (NZ) LIMITED, Auckland (NZ)

(21) Appl. No.: 17/217,949

(22) Filed: Mar. 30, 2021

(30) Foreign Application Priority Data
Mar. 31, 2020 (NZ) 762953

(51) Int. Cl. A61F 13/15

(52) U.S. Cl. CPC A61F 13/15585; A61F 2013/49098; A61F 13/15252

(57) ABSTRACT

Described herein is a disposable diaper that is substantially bio based and/or biodegradable. The diaper comprises a biodegradable top sheet, a biodegradable super absorbent core and biodegradable water resistant outer layer that is liquid impervious and a breathable bottom sheet, wherein the top sheet is configured for transmission of bodily fluids to the super absorbent core. The absorbent core comprises a first and second layer comprising pulp fibres. The diaper is either manufactured of wholly or partially renewable resources, at the same time can substantially decompose in a compost site, and substantially biodegrade in a conventional landfill.

This patent application is a Paris Convention filing of NZ762953, filed 31 Mar. 2020, the specification of which is hereby incorporated herein by reference.

### BACKGROUND OF THE INVENTION

### Field of the Invention

Described herein is a disposable diaper that is substantially bio-based and/or biodegradable. The diaper comprises a biodegradable top sheet, a biodegradable super absorbent core and biodegradable water resistant outer layer that is liquid impervious and a breathable bottom sheet, wherein the top sheet is configured for transmission of bodily fluids to the super absorbent core. The absorbent core comprises a first and second layer comprising pulp fibres. The diaper is either manufactured of wholly or partially renewable resources, at the same time can substantially decompose in a compost site, and substantially biodegrade in a conventional landfill.

### Description of the Related Art

For many years cloth diapers were used over and over again, being washed between each use. However, for consumer convenience single use disposable diapers were introduced to the market. Disposable diapers generally consisted of a liquid impervious plastic back sheet, an absorbent pad, and a liquid permeable plastic top sheet.

As above, such disposable diapers were designed to become discarded after a single use and have become a major source of waste. A baby may use 6-8 diapers a day, leading up to more than 2000 diapers per year. Such disposable diapers cannot be recycled successfully because of the large amount of cellulose and other materials present in the diaper. Disposal of such diapers in landfills contributes to the accumulation of garbage. The plastic materials do not break down under landfill conditions and remain in their original form for hundreds of years.

Attempts have been made to alleviate this problem by using plastics which are “biodegradable”. U.S. Pat. No. 5,185,009 describes using a biodegradable polyethylene. Polyethylene can be biodegradable in landfill, where there is no UV exposure and only if it is copolymerised with a “truly biodegradable” polymer such as starch.

In light of the above, new compostable diapers have been designed and marketed. For example, an Eco nappy manufactured by Little & Brave comprising a disposable pad suitable for commercial composting and having a biodegradable cellulose based outer cover (rayon), and inside for absorbency they have non-chlorine bleached fluff (paper) pulp and a super absorbing polymer (SAP). A disadvantage of this nappy is that disposable pad requires commercial composting and has to be taken to a particular off-site drop off point for disposable. Furthermore, the nappy comprises a reusable washable fabric that requires separate washing to the disposable pad.

TwinkleBotts also manufacture a disposable nappy with a cotton based back sheet where the majority of materials used in the nappy are composed of natural fibres. It features a chemical blend of absorbent polymers (SAP).

Another such compostable diaper is known from EP0525245A1. It is observed therein that a conventional disposable absorbent product consists of about 80% of compostable, such as wood pulp fibres. In the composting process, the articles are shredded and co-mingled with organic waste prior to the composting per se. After composting is complete, the non-compostable particles are screened out. Since the amount of non-compostable particles was too high, EP0525245 disclosed a product with a liquid impervious back sheet comprising a compostable polymer. This back sheet thereto contained a flexible starch based film. The preferred top sheet contained staple-length propylene fibres with a length of at least about 15 mm. A more recent embodiment of such a biodegradable liquid impervious back sheet, comprising a laminate of films, is for instance known from WO 2013/137817.

Biodegradable and compostable liquid permeable top sheets are also known. Alternatively, use can be made of biodegradable, liquid impervious top sheets with one or more openings.

Still, improvements to compostable diapers are desired. Particularly, composting of diapers will only make sense on a large scale if the composting process does not require commercial intervention; i.e. any processing on the diapers such as shredding and combing them with other materials, tends to lower economic viability due to labour costs.

From the above, it can be seen that there is a requirement to provide an improved disposable diaper that is substantially biodegradable and compostable in a manner that is economically viable and/or at least provides the public with a useful choice. Further aspects and advantages of the composition of the product, manufacture and uses thereof—will become apparent from the ensuing description that is given by way of example only.

### BRIEF SUMMARY OF THE INVENTION

Described herein is a disposable diaper that is bio-based and/or biodegradable. The diaper comprises a biodegradable top sheet, a biodegradable super absorbent core and biodegradable water resistant outer layer that is liquid impervious and a breathable bottom sheet, wherein the top sheet is configured for transmission of bodily fluids to the super absorbent core. The absorbent core comprises a first and second layer comprising pulp fibres. The diaper is either manufactured of wholly or partially renewable resources, at the same time can substantially decompose in a compost site, and substantially biodegrade in a conventional landfill.

In a first aspect there is provided a diaper comprising a biodegradable top sheet, a biodegradable super absorbent core and biodegradable water resistant outer layer that is liquid impervious and a breathable bottom sheet, wherein the top sheet is configured for transmission of bodily fluids to the super absorbent core, wherein the absorbent core comprises a first and a second non-woven layer comprising pulp fibres, and wherein the diaper materials are at least 70 wt. % biodegradable and at least 90 wt. % compostable.

In a second aspect there is provided a diaper comprising a biodegradable top sheet, a biodegradable super absorbent core and biodegradable water resistant outer layer that is liquid impervious and a breathable bottom sheet, wherein the top sheet is configured for transmission of bodily fluids to the super absorbent core, wherein the absorbent core comprises a first and a second non-woven layer comprising pulp fibres, and wherein the diaper materials are at least 90% biodegradable by volume (v/v %) and at least 90 wt. % compostable.

In a third aspect there is provided a method of manufacturing a diaper as substantially described above.

Advantages of the above include an eco-friendly nappy that on average only requires two pieces of material per day, with at least 94 wt. % home compostability with reduced impact on landfills of 10 g (2×10%×50 g=10 g). This compares with a traditional disposable nappy that on average utilises 5 pieces of material per day, with 15 wt. % biodegradability (pulp) with an impact on landfills of 212.5 g (5×85%×50 g). Note that these figures are based on an average nappy weight of 50 g. The majority of the “eco-nappies” on the market currently at best are only commercially compostable. A key advantage of this invention is that the nappy is at least 90 wt. % and/or 90% by volume biodegradable and 94 wt. % home compostable which means that there is no longer an issue for local authorities and the problem of disposal can be achieved at a user's home. That is, the nappy can be buried in a home compostable bin where the non-toxic materials can be broken down after 45 days. Besides reduced environmental impact, the nappy minimises leakage and does not break apart compared to traditional “eco-nappies” owing to the key combination and selection of materials. Furthermore, the nappy includes a 100% compostable outer bag i.e. a non-plastic based nappy packaging which increase the total biodegradability of the nappy package.

### BRIEF DESCRIPTION OF THE DRAWINGS

Further aspects of the composition of the product, manufacture and uses thereof will become apparent from the following description that is given by way of example only and with reference to the accompanying drawings in which:

FIG. 1 illustrates a perspective view of an exemplary nappy embodiment;

FIG. 2 illustrates a component breakdown of the nappy embodiment of FIG. 1 and compostability and biodegradability (wt. %); Newborn (2a); Infant (2b); Crawler (2c); Toddler (2d); Walker (2e) and Junior (2f);

FIG. 3 illustrates a component breakdown of the nappy embodiment of FIG. 1 and compostability and biodegradability (volume %); Newborn (3a); Small (3b); Medium (3c); Large (3d); Xtra Large (3e); and Xtra Xtra Large (3f);

FIG. 4 illustrates a perspective view of an exemplary nappy pant embodiment;

FIG. 5 illustrates a component breakdown of the nappy pant embodiment of FIG. 4 and compostability and biodegradability (wt. %); Toddler (5a); Walker (5b); and Junior (5c);

FIG. 6 illustrates a component breakdown of the nappy pant embodiment of FIG. 4 and compostability and biodegradability (volume %); Large (6a); Xtra Large (6b); and Xtra Xtra Large (6c);

FIG. 7 illustrates an exemplary compostable multi-layer outer bag used for packaging of the nappy embodiment; and

FIG. 8 illustrates the component breakdown of the constituent SAP mix.

### DETAILED DESCRIPTION OF THE INVENTION

As noted above, described herein is a disposable diaper that is substantially bio based and/or biodegradable. The diaper comprises a biodegradable top sheet, a biodegradable super absorbent core and biodegradable water resistant outer layer that is liquid impervious and a breathable bottom sheet, wherein the top sheet is configured for transmission of bodily fluids to the super absorbent core. The absorbent core comprises a first and second layer comprising pulp fibres. The diaper is either manufactured of wholly or partially renewable resources, and at the same time can substantially decompose in a compost site, and substantially biodegrade in a conventional landfill.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refers to at least about 50%, for example 75%, 85%, 95% or 98%.

The term ‘comprise’ and grammatical variations thereof shall have an inclusive meaning—i.e. that it will be taken to mean an inclusion of not only the listed components it directly references, but also other non-specified components or elements.

The term ‘diaper’ refers to sanitary articles that allows the wearer to urinate or defecate without the use of a toilet and may be used interchangeably throughout the specification with nappy and/or nappy pant. Sanitary articles may include, but should not be seen as limited to, disposable diapers, adult incontinence pads, feminine hygiene products, and/or sanitary napkins comprised of biodegradable polymers with higher bio-based content.

The term ‘bio-based’ or grammatical variations thereof refers to a material, composition and/or substance that is derived, produced or synthesised in part or in whole from a renewable source such as a plant or animal source. For example, polylactic acid (PLA) which is produced by chemical and/or enzymatic conversion of corn starch is a bio-based polyester. Similarly, polyhydroxyalkanotes such as polyhydroxybutyric acid (PUB) are polyesters produced by bacterial or enzymatic conversion of carbohydrates such as sugar or glucose or canola oil.

The term ‘compostable’ or grammatical variations thereof refers to a carbon based material that can be bacterially, enzymatically and/or hydrolytically degraded under conditions typically present in organic composting processes. Such conditions usually involve warm temperatures, a combination of aerobic and anaerobic bacterial activity and non-bacterial hydrolysis. Typically, a compostable material will be decomposed by bacterial and/or enzymatic action and/or by hydrolysis to a point where the compostable material is no longer recognizable or distinguishable from humus. However, the compostable material typically is not converted at this point to carbon dioxide and water plus inorganic residue.

The term ‘biodegradable’ or grammatical variations thereof refers to a carbon based material that can be bacterially and/or enzymatically decomposed under conditions that are typically present in landfills. Such conditions usually include deep burial so that anaerobic bacterial and/or enzymatic activity typically will be the primary mode of decomposition. Such conditions typically do not include substantial non-bacterial hydrolysis but hydrolysis may occur to some extent. Typically, the biodegradable material is converted primarily, substantially, essentially or completely to ultimate decomposition products such as carbon dioxide, water and inorganic residue. The bacterial and/or enzymatic decomposition will begin shortly after burial.

It should be appreciated that while all compostable material is biodegradable, not all biodegradable material is compostable. Although biodegradable materials return to nature and can disappear completely they sometimes leave behind inorganic residue, on the other hand, compostable materials create humus as aforementioned that is full of nutrients and great for plants. Compostable products are biodegradable, but with an added benefit. That is, when they break down, they release valuable nutrients into the soil, aiding the growth of trees and plants.

The term ‘liquid impermeable’ or grammatical variations thereof refers a substance formed as a film or sheet that will not allow water and/or an aqueous medium to pass through it.

Such a substance may exhibit leakage such that a miniscule amount of water or aqueous medium may eventually make its way through the substance over a significant period of time such as 2 to 6 hours or longer. Preferably, miniscule leakage is also not present. Such a substance may allow air to pass through but air permeability is not a requirement.

The term ‘liquid permeable’ or grammatical variations thereof refers to a substance formed as a film or sheet will allow water and/or and aqueous medium to pass through it. The permeability may be the result of pores or may be the result of a non-porous, but water diffusible material. Water diffusion through such a permeable material is typically passive.

The term ‘superabsorbent’ or grammatical variations thereof refers to a substance that absorbs aqueous medium is able to take on a significant weight proportion of aqueous medium relative to its dry weight. Preferably, such a substance has a dry feel even though it has absorbed a significant weight portion of aqueous medium.

The term ‘elastic’ or grammatical variations thereof refers to a substance can be stretched and/or deformed under stress and will return to its original shape, size and consistency upon discontinuation of the stress.

The term ‘polyester’ refers to a polymer in which monomer units are linked together by ester (—COO—) groups, usually formed by polymeric esterification of a diol and a diacid or esterification of a hydroxyacid. Examples of the hydroxyacid polyester include polylactic acid (PLA), polyglycolic acid (PLG), polylactic/glycolic acid (PLG), polyhydroxybutyric acid (PHB), polyhydroxypropanoic acid. The polyester may also or alternatively be analiphatic polyester, an aromatic polyester of a combination thereof. Examples ofthe diacid polyesters include polybutylene succinate (PBS) and polyhexyleneadipate (PHA). Aromatic polyesters are typically and usually formed by condensation of aliphatic diols and aromatic dicarboxylic acids. Typical production techniques include terphthalate substitution for the diacid or inclusion of terphthalate along with the aliphatic diacid. Examples include polybutyleneadipate terephthalate (PBAT), polybutylenesuccinate terphthalate, polyhexylenesuccinate terphthalate, polybutylene terphthalate, polyhexylene terphthalate, polybutyleneadipate terphthalate, polyhexylene adipate terphthalate.

The term polyolefin means a polymer formed from an organic compound having at least one carbon-carbon double bond that is not aromatic, with an empirical formula CnH₂n. Examples include polyethylene, polypropylene, polybutylene and polyisobutylene. A polyolefin may be bio-based or petroleum based. Rubber or rubber band is a typical generic name for vulcanized polyisobutylene, which is polyisobutylene cross-linked with sulphur.

The term aqueous medium in the context of the invention means human or mammal urine, faeces and similar human or mammal excrement.

In a first aspect there is provided a diaper comprising a biodegradable top sheet, a biodegradable super absorbent core or water resistant sheet that is liquid impervious and a breathable bottom sheet, wherein the top sheet is configured for transmission of bodily fluids to the super absorbent core, wherein the absorbent core comprises a first and a second non-woven layer comprising pulp fibres, and wherein the diaper materials are at least 70 wt. % biodegradable and at least 90 wt. % compostable.

As above, the disposable diaper may be constructed primarily, substantially or essentially of bio-based and/or biodegradable materials such that the disposable diaper may decompose at least 90 wt. % in a moderate amount of time in a landfill, in a burial site, in a compost site and/or in a garbage dump. Typically, the decomposition of the disposable diaper may produce humus and residual material that is not recognisable as diaper material. The humus and residual material may be of a chemical nature that may enable them to be assimilated by resident organisms present in soil, landfills, compost sites and dumps.

Preferably, the decomposition may result primarily, substantially or essentially in further reduction of the diaper materials into carbon dioxide, water and inorganic residue.

As above, preferably the diaper materials may be at least 70% biodegradable by weight (wt. %) and/or at least 90% compostable by weight (wt. %). Alternatively, as measured by volume percent (v/v %), preferably the diaper materials may be at least 90% biodegradable and/or compostable by volume. The biodegradability and compostability may range substantially from 70, or 75, or 80, or 85, or 86, or 87, or 88, or 89, or 90, or 91, or 92, or 93, or 94, or 95, or greater (wt. %) and/or (v/v %).

These embodiments of the disposable diapers of the present invention preferably include a breathable back sheet outer sheet made of 100% pure cotton which may be compostable and biodegradable by at least 8 wt. % respectively.

The diapers of the foregoing embodiments may alternatively or additionally include an impermeable and or water resistant sheet made of biodegradable aromatic polyester. Preferably, the biodegradable aromatic polyester is polylactic acid (PLA) derived from a plant source, preferably corn which may be compostable and biodegradable by at least 15 wt. % respectively.

Alternatively, the embodiments of the diapers of present invention include a top sheet that is also made of an aromatic polyester is polylactic acid (PLA) derived from a plant source, preferably corn which may be compostable and biodegradable by at least 7 wt. % respectively. In this way, the corn starch soft top sheet may be gentle on developing and delicate skin.

The sheets may be coated with a layer of cellulose fibre such as paper to improve the mechanical property or may be printed on with graphical designs and the like using a plant based biofilm with water based inks.

These embodiments of the disposable diapers of the present invention preferably include an inner core comprising first and second non-woven layers. The first non-woven layer may comprise a core wrap component. The core wrap component may be tissue paper which locks wetness away and keeps skin dry. The non-woven layers preferably contain a fluff material. Fluff material are known as fibres that typically have a moisture content of less than 10% and are of a hydrophilic nature. Fluff pulps are typically processed into non-wovens by means of dry-laying processes, such as air laying, spunbonding, hydroentangling. Suitably, the non-wovens are dried in a continuous sheet form and wound onto rolls. It will be understood that the pulp fibres are the primary elements of the non-woven layers. In preferred embodiments, the pulp is Forest Stewardship Council (FSC) certified fluff wood pulp which may be compostable and biodegradable by at least 27 wt. % respectively.

Suitably, the first non-woven layer, which is arranged adjacent to the top sheet, comprises fibre particles (the fluff) with an average particle size that is smaller than the second-non-woven layer. The smaller particle size of the first-non woven layer is found to contribute to distribution of urine and/or other bodily fluids through the diaper. The larger particle size of the particles in the second non-woven layer (wood pulp) is deemed beneficial to increase moisture absorption. This, in this manner, urine is removed from the user's skin quickly. In one suitable embodiment, the average particle size in the first non-woven layer is less than 3 mm, suitably at most 2 mm, preferably at most 1 mm.

More preferably, in combination with the non-woven layers there may be a superabsorbent polymer (SAP). A superabsorbent polymer (SAP) is a polymer that absorbs from several to hundreds of times its own weight in water. It may be a high molecular weight polymer featuring water absorption and retention properties e.g. by turning into a gel significantly improving nappy absorbency while maintaining an extremely stable form.

In one embodiment, the SAP may be AQUA KEEP™ a Japanese super absorbent polymer manufactured by Sumitomo Seika comprising mainly of sodium polyacrylate, wherein the saturation ratio may be 90% compared to standard SAP's with 75%. It has been found that this is an efficient compost additive with an extremely high water retention functionality. AQUA KEEP™ has a high content of —COO— and Na+ ions. The difference between the ion concentration inside the polymer and that of the surrounding water solution determines the intensity of osmotic pressure. The lower the ion concentration of the surrounding water solution, the greater the resultant difference in ion concentration, and accordingly the osmotic pressure goes up. This osmotic pressure enables the polymer to absorb a large quantity of water.

The affinity of the polymer with its surrounding solution also affects the absorption capacity of the polymer. The hydrophilic groups “—COOH” and “—COONa” in AQUA KEEP™ have a high affinity for water, allowing a water solution to be absorbed. The affinity, however, is less significant than osmotic pressure in determining the polymer's absorption capacity.

As a result of these two factors, the polymer may continue to absorb water to the extent that the ion concentration between inside the polymer and the surrounding solution equalizes. To control the water absorption to an intended level, the polymer must be provided with specific rubber elasticity.

The rubber elasticity of the polymer increases as the crosslinking density of that polymer increases. The absorption capacity of the polymer reaches its maximum when its rubber elasticity and its water absorbing power generated from osmotic pressure and affinity of the polymer are balanced.

The proportion or ratio of SAP to fluff pulp material mix may range from 1:0.5-2.5, although this ratio should not be seen as limiting as other key ratios could conceivably be used with this invention. It has been found that a key inventive aspect of this invention is the combination of the key materials. In particular, the SAP ratio and mix needs to the right amount to absorb the liquid quick enough and overcome the difficulties associated with the other plant based cover materials. For example, if the incorrect portion or ratio of SAP is utilised, the nappy would not absorb enough urine etc. Likewise, if too much SAP is utilised then the nappy is not as effective for composting/biodegradability. Furthermore, the inventor has unexpectedly found that the proportion or ratio of Sumitoto AQUA KEEP™SAP to other sandia SAP when combined also provides the advantages as described above. In preferred embodiments the proportion or ratio of Sumitoto AQUA KEEP™ SAP to sandia SAP may range from 6:4, although this ratio should not be seen as limiting as other key ratios could conceivably be used with this invention. Without being bound by theory, again it has been found that a key inventive aspect of this invention is the combination of the key materials. In particular, the combination of SAP ratios needs to be the right amount to absorb the liquid quick enough and overcome the difficulties associated with the other plant based cover materials.

As an alternative embodiment of the disposable diapers of the present invention, the superabsorbent fillers may be starch based. Preferably, the superabsorbent has a bio-based content greater than 25%. Alternatively, the superabsorbent is protein based. Alternatively, the superabsorbent is keratin based.

Further embodiments of the disposable diaper of the present invention include a 3D core, side sheet, leak guard and/or double leak guards made of a non-woven aliphatic polyester. Preferably, the side sheets are configured with 3D elastic that may automatically position themselves such that there is no requirement to manually pull them out. In this way, the core increases the absorbency area, creating absorbency channels down the side of the core to prevent further leaks. Optionally, the leak guard may be made of an aliphatic polyester that is PHA. Preferably, the PHA is bio-based and the bio-based content is greater than 25%. Alternatively, the side sheets are made of an aliphatic polyester that is PBS or its copolymers PBSA. Preferably, the PBS or PBSA is bio-based and the bio-based content is greater than 25%.

As another alternative, the side sheets aremade of biodegradable aromatic polyester. Preferably, the biodegradable aromatic polyester is PBAT. As a further alternative, the side sheets are made of cellulosic fibre treated with a biodegradable polymer. As another alternative, the side sheets are made of a biodegradable polymer thin film laminated on a biodegradable non-woven material. Preferably, the biodegradable nonwoven material is made of polylactic acid. As a further alternative, the embodiments of the bio-based and biodegradable disposable diapers of the present invention include side sheets made of a combination of aliphatic polyester and aromatic polyester. Preferably, the combination is bio-based and the bio-based content greater than 25%.

The embodiments of the bio-based and/or biodegradable disposable diapers of the present invention include fastening grip tabs with an S-shape. In this way, they are smoother on skin and prevent unnecessary damage.

This grip tabs are made from a combination of polypropylene and non-woven materials. In alternative embodiments, the grip tabs may be made from a combination of aliphatic polyester and aromatic polyester that may be bio-based and wherein the bio-based content may be greater than 25%.

As an alternative, the fastening tabs are made of polypropylene. Preferably, the polypropylene is derived from plant material such as corn or soybeans. Preferably, the polypropylene is bio-based and the bio-based content is greater than 25%. As an alternative, the fastening tabs are made of polylactic acid.

The diaper may include a fastening frontal tape portion of a combination of cotton blended with polypropylene and polyethylene material. In this way, the material is ultra-soft and gentle on delicate skin. As described in embodiments above, it is envisaged that the frontal tape portion may be made of bio-based material and the bio-based content is greater than 25%.

The embodiments of the disposable diapers of the present invention include an elastic waistband that may ensure a comfortable fit around the waist and helps to prevent leakage from the back. It may be made of a combination of polylactic acid (PLA) derived from a plant source, preferably corn and cotton. It is envisaged that the elastic waistband may be made of a bio-based and the bio-based content is greater than 25%. As an alternative, the elastic waistband may be made of a of aliphatic polyester and aromatic polyester.

The embodiments of the disposable diapers of the present invention include a soft leg cuff to provide a comfortable fit around the legs constructed of natural fibre material. Preferably, the natural fibre is cotton. Alternatively, the natural fibre is bamboo fibre.

In yet further embodiments, the nappy when sold as a package may include a 100% compostable outer bag. In this way, the packaging contents are non-plastic based which increases the total nappy biodegradability package to over 90 wt. %. It is envisaged that the compostable multi-layer outer bag may confirm with the compostable regulations (industrial) of E.U. and U.S.A, food contact grade, using environmentally-friendly materials. This type of packaging is mainly used for packaging with no liquid, temperature below 50° C., with no requirement of oxygen or waterproofing applications.

The types of outer bags may include, but not be seen as limited to three-side seal bags, side-gusset bags, stand-up pouch, and quad pouches etc.

It is envisaged that the compostable multi-layer outer bag may have at least a 1 year shelf life under dry and ventilated conditions.

The length and width of the multi-layer outer bag may range from length (50 mm to 1000 mm) to width (50 mm to 600 mm) with a 3″ core roll diameter (95 mm to 600 mm) and width (20 mm to 1200 mm).

The structure of the compostable multi-layer outer bag may comprise, but should not be seen as limited to paper (or cellulose), adhesive or PLA with a sealing range of 135−170° C.

In a second aspect there is provided a diaper comprising a biodegradable top sheet, a biodegradable super absorbent core and biodegradable water resistant outer layer that is liquid impervious and a breathable bottom sheet, wherein the top sheet is configured for transmission of bodily fluids to the super absorbent core, wherein the absorbent core comprises a first and a second non-woven layer comprising pulp fibres, and wherein the diaper materials are at least 90% biodegradable by volume and at least 90 wt. % compostable.

In a third aspect there is provided a method of manufacturing a diaper as substantially described above.

The embodiments of the disposable diapers of the present invention are produced by formation of the outer sheet, inner sheet, or the side sheets by a spun bond method. Alternatively, the outer sheet, inner sheet, or the side sheets are formed by a thermal bond method. Alternatively, the outer sheet, inner sheet, or the side sheets are formed by a stitch bond method. As a further processing feature, the inner sheet is formed by a needle punch method. As a further processing feature, the outer sheet is formed by a melt blown method.

In all of the foregoing embodiments, the combination of materials may be incorporated as the inner and outer sheets, side sheets, tabs, and elastic waistband etc.

The embodiments described above may also be said broadly to consist in the parts, elements and features referred to or indicated in the specification of the application, individually or collectively, and any or all combinations of any two or more said parts, elements or features.

Further, where specific integers are mentioned herein which have known equivalents in the art to which the embodiments relate, such known equivalents are deemed to be incorporated herein as if individually set forth.

### WORKING EXAMPLES

The above described composition of the products, manufacture and uses thereof are now described by reference to specific examples.

### Example 1

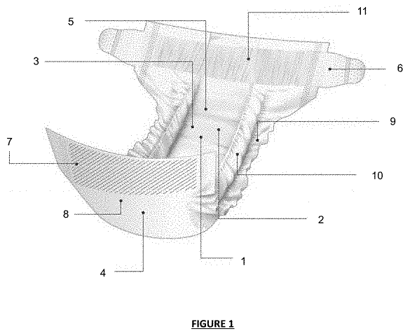

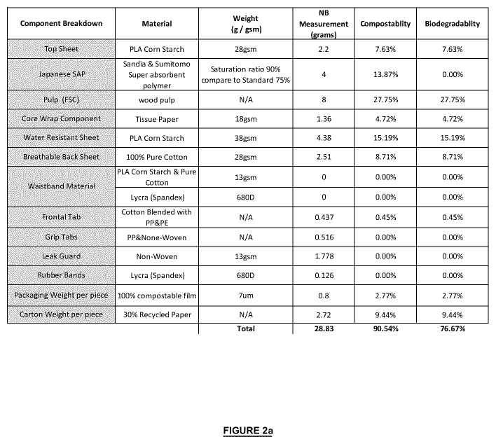

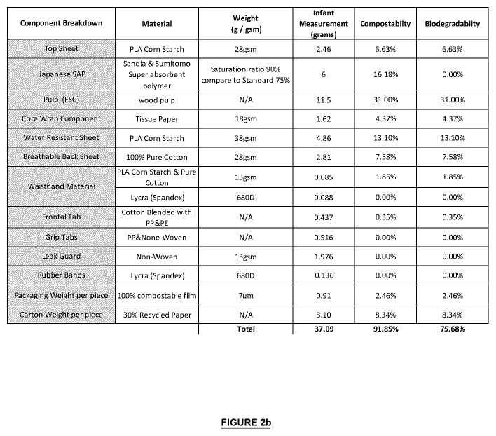

FIG. 1 illustrates each component of a disposable diaper or nappy. While FIG. 1 illustrates an embodiment of the disposable diaper according to the invention, other configurations of a disposable diaper constructed primarily, substantially or essentially of bio-based material with the option of inclusion of some petroleum based material will be clearly apparent to the skilled practitioner.

As illustrated in FIG. 1, the inner layers i.e. an absorbent aqueous permeable membrane comprising a core wrap component 1, wood pulp 2 and SAP 3 which allows aqueous liquid and other excrements to pass through to the absorbent pad underneath. The inner layers such as the core wrap component 1 for locking wetness away and keeping the baby's skin dry is made of tissue paper, wood pulp 2 (100% natural material FSC certified pulp) and super absorbent polymers 3 (safe for the planet as an effective compost additive with an extremely high water retention) respectively.

The outer sheet is aqueous impermeable comprising a water resistant sheet 4. It is made of bio-based and/or biodegradable material that is a 100% biodegradable non-GMO corn starch based PLA. The top sheet 5 is also 100% biodegradable manufactured out of corn starch and is soft and gentle on developing and delicate skin.

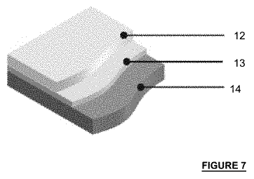

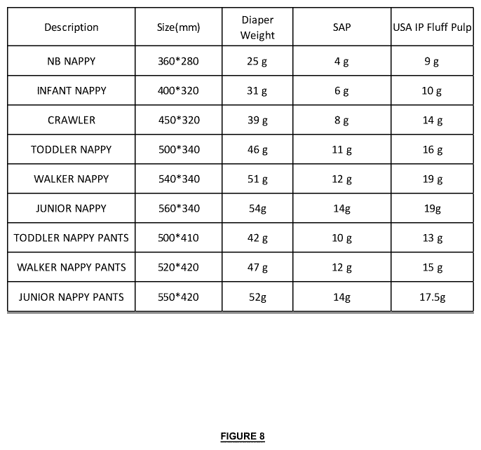

As above, the absorbent pad is a blend of natural fibres and a superabsorbent core. The natural fibre can be cellulosic such as wood pulp fibre. The preferred superabsorbent has a saturation ratio of greater than 90%. The proportion or ratio of SAP 3 to pulp is 1:0.5-2.5 and is shown in FIG. 8.

The illustrated embodiment includes fastening grip tabs 6 at two corners. The tabs 6 are a “S-Shape” design to ensure smoothness on a baby's skin and prevent any unnecessary damage. The tabs 6 can be attached to the frontal tape portion 7 on other end of the diaper by an adhesive type connection to secure the diaper in position on an infant. The frontal tape 7 is attached to a breathable back sheet 8 manufactured from a cotton blend which is ultra-soft and gentle on delicate skin. It is envisaged that in future embodiments, both the fastening tabs and the frontal tape portion are made of bio-based and/or biodegradable material. In this embodiment, the tear tabs 6 and frontal tapes 7 are torn off prior to disposal and recycling.

The illustration in FIG. 1 showing that the diaper has a soft leg cuff section 9 manufactured out of 100% cotton which fits the leg of an infant to provide for a comfortable fit around the legs. It is desirable to include elastic side sheets or leak guards 10 to avoid leakage. The side sheets or leak guards 10 are aqueous impermeable and are specially designed 3D elastic which automatically position themselves without the requirement of needing to pull them out. Attached to side sheets are bio-based and/or biodegradable rubber bands, making the sides sheets elastic.

For a diaper to hold comfortably tight on a body such as that of an infant, an elastic waist band 11 comprising cotton and corn starch is attached to the diaper to ensure a comfortable fit around the baby's waist and helps prevent leakage from the back. It is envisaged that the preferred bio-based content of the elastic waist strip is greater than 25%. The inner sheet, outer sheet, and the side sheets are made of biodegradable aliphatic polyester materials derived from bio-based and/or renewable sources. Bio-based polyhydroxyalkanoates and bio-based polybutylenesuccinate are examples of such biodegradable material. The sheets can be formed by spun bond method, thermal bond method, stitch bond method, needle punch method, or a melt blown method. These sheets can also be coated with a layer of cellulose fibre such as paper to improve the mechanical property or be printed on with graphical designs and the like using a plant based biofilm with water based inks.

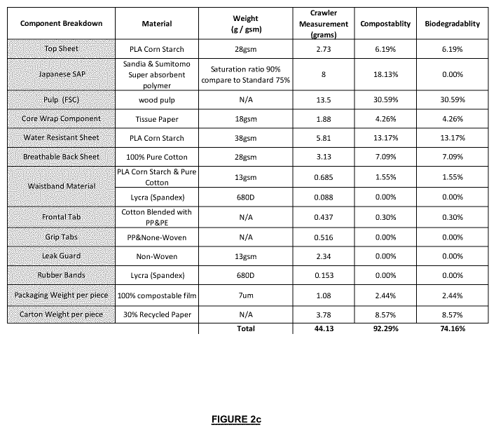

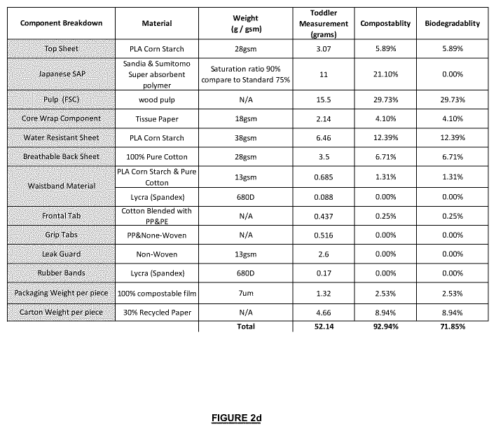

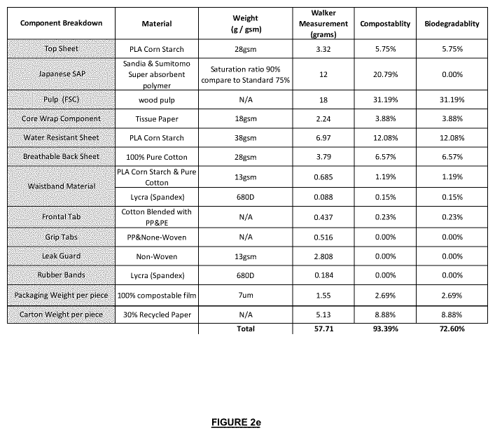

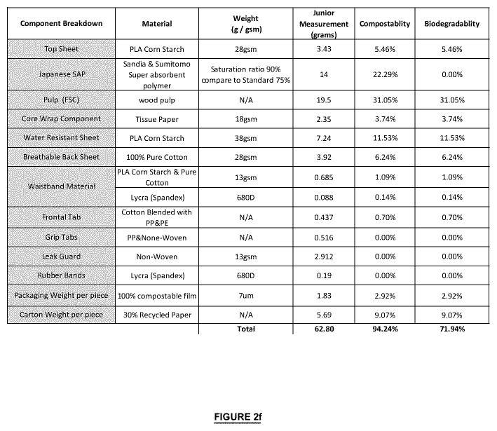

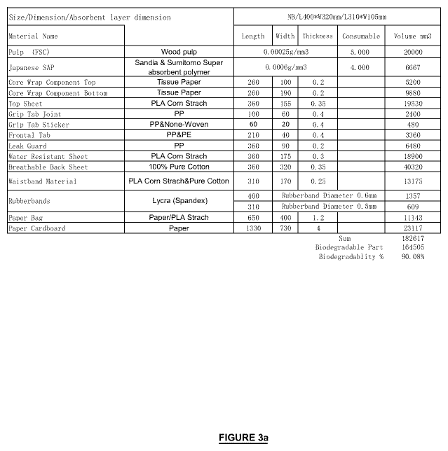

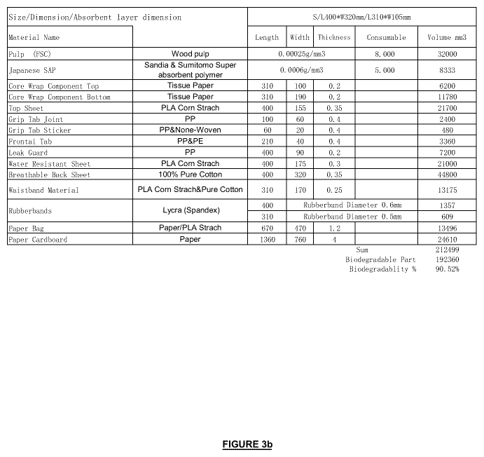

At the present time, it should be noted that 90-94% of the materials are home compostable and FIGS. 2 and 3 respectively shows the component breakdown of the nappy embodiment of FIG. 1. In particular, FIG. 2 illustrates the compostability and biodegradability of each component and combined total measured as wt. % for the varying nappy sizes designated as Newborn (FIG. 2a); Infant (FIG. 2b); Crawler (FIG. 2c); Toddler (FIG. 2d); Walker (FIG. 2e) and Junior (FIG. 2f).

FIG. 3 illustrates a further component breakdown of the nappy embodiment of FIG. 1.

In particular, FIG. 3 illustrates the compostability and biodegradability of each component and combined total measured as volume % for the varying nappy sizes designated as; Newborn (3a); Small (3b); Medium (3c); Large (3d); Xtra Large (3e); and Xtra Xtra Large (3f).

It is further envisaged that the inventors may achieve closer to 100% home compostability and overcome the difficulties associated with materials that are less compostable.

### Example 2

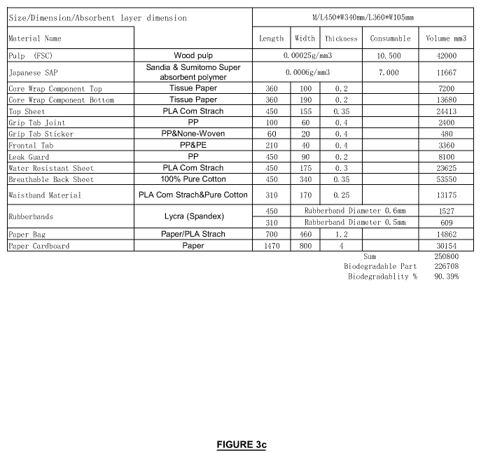

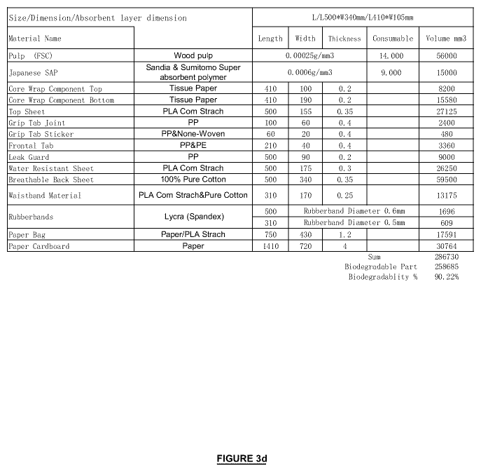

FIG. 4 illustrates each component of a disposable nappy pant and includes the same features as described for the nappy embodiment of FIG. 1 (but without tabs 6 and frontal tape portion 7) comprising: a core wrap component 1, wood pulp 2, SAP 3, an aqueous impermeable outer sheet 4, top sheet 5, frontal tape portion 7, breathable back sheet 8, soft leg cuff section 9, elastic side sheets or leak guards 10 and an elastic waist band 11.

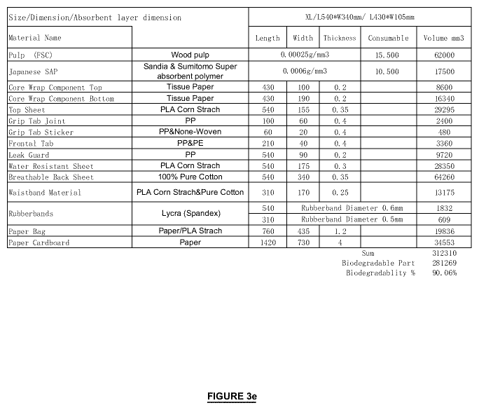

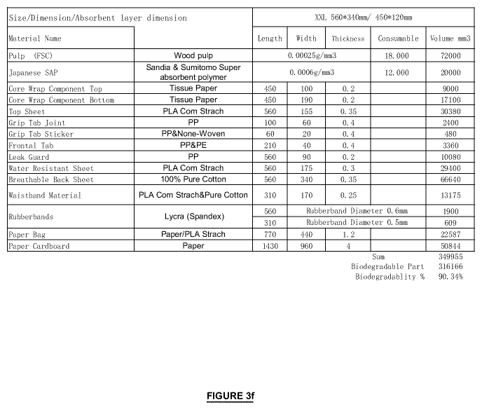

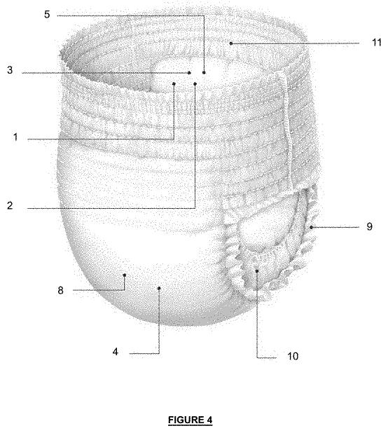

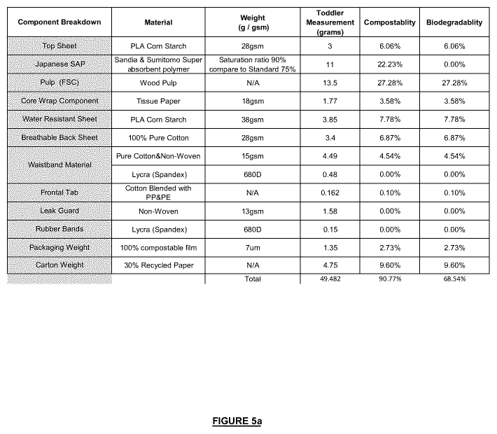

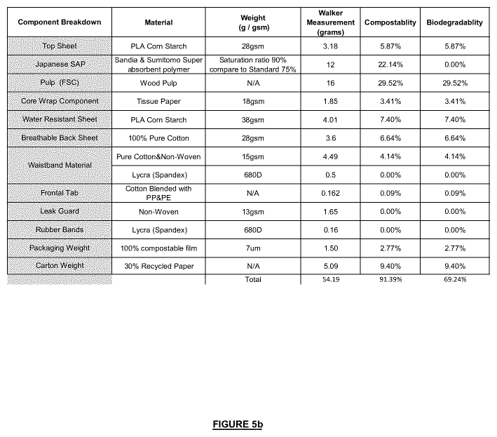

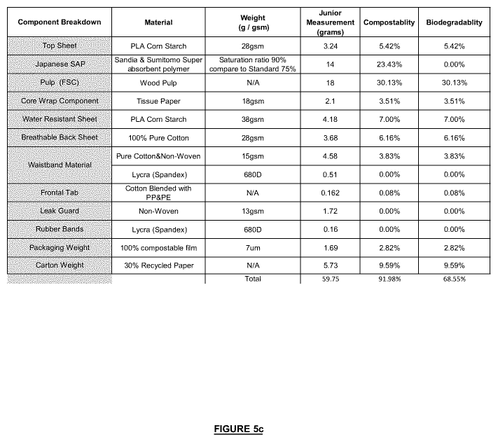

FIGS. 5 and 6 respectively shows the component breakdown of the nappy pant embodiment of FIG. 4. In particular, FIG. 5 illustrates the compostability and biodegradability of each component and combined total measured as wt. % for the varying nappy pants designated as Toddler (FIG. 5a); Walker (FIG. 5b); and Junior (FIG. 5c).

FIG. 6 illustrates a further component breakdown of the nappy pant embodiment of FIG. 4. In particular, FIG. 6 illustrates the compostability and biodegradability of each component and combined total measured as volume % for the varying nappy pants designated as Large (FIG. 6a); Xtra Large (FIG. 6b); and Xtra Xtra Large (FIG. 6c)

### Example 3

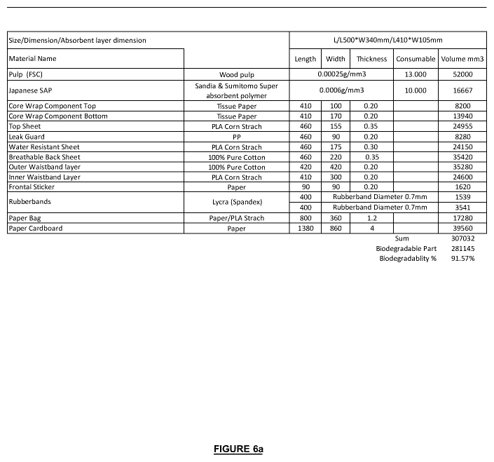

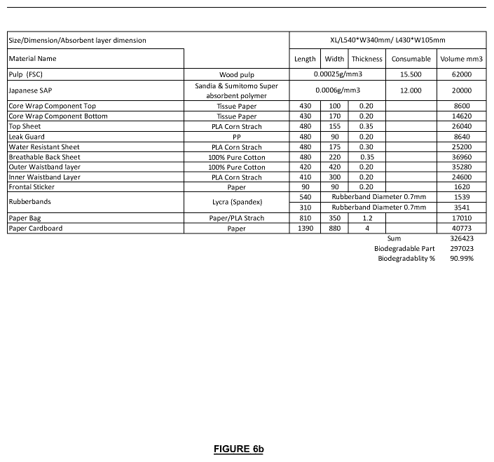

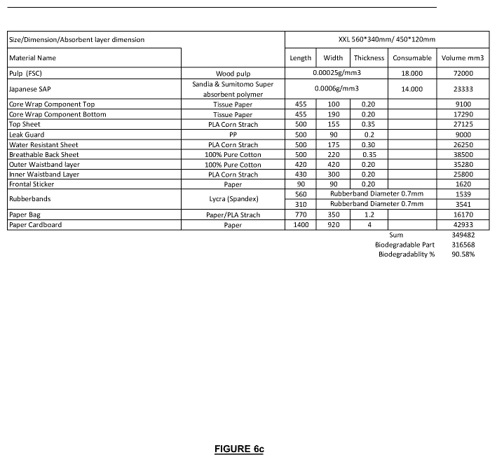

With reference to FIG. 7, this illustrates each component of a compostable multi-layer outer bag used as packaging for the disposable nappy and nappy pant when sold comprising a printing layer 12 manufactured out of paper/cellulose, a tie layer or compostable adhesive 13 and a sealing layer 14 manufactured out of PLA with a sealing range of 135−170° C.

In this way, the packaging contents are non-plastic based which increases the total nappy biodegradability package to over 90 wt. %. It is envisaged that the compostable multi-layer outer bag may confirm with the compostable regulations (industrial) of E.U. and U.S.A, food contact grade, using environmentally-friendly materials. This type of packaging is mainly used for packaging with no liquid, temperature below 50° C., with no requirement of oxygen or waterproofing applications.

The compostable multi-layer outer bag has at least a 1 year shelf life under dry and ventilated conditions.

The length and width of the multi-layer outer bag ranges from length (50 mm to 1000 mm) to width (50 mm to 600 mm) with a 3″ core roll diameter (95 mm to 600 mm) and width (20 mm to 1200 mm).

Aspects of the present invention have been described by way of example only and it should be appreciated that modifications and additions may be made thereto without departing from the scope of the claims herein.

What is claimed is:

1. A diaper comprising diaper materials that comprise a biodegradable top sheet, a top sheet, a biodegradable super absorbent core or water resistant sheet that is liquid impervious and a breathable bottom sheet, wherein the top sheet is configured for transmission of bodily fluids to the biodegradable super absorbent core, wherein the biodegradable super absorbent core comprises a first and a second non-woven layer comprising pulp fibres, and wherein the diaper materials are at least 70 wt. % biodegradable and at least 90 wt. % compostable.

2. The diaper as claimed in claim 1, wherein the diaper is constructed primarily, substantially or essentially of bio-based and/or biodegradable materials such that the diaper decomposes at least 90 wt. % in a moderate amount of time in a landfill, in a burial site, in a compost site and/or in a garbage dump without further commercial processing.

3. The diaper as claimed in claim 1, wherein decomposition results primarily, substantially or essentially in further reduction of the diaper materials into carbon dioxide, water and inorganic residue.

4. The diaper as claimed in claim 1, wherein a breathable back sheet outer sheet is manufactured of 100% pure cotton and is compostable and biodegradable by at least 8 wt. % respectively.

5. The diaper as claimed in claim 1, wherein the water resistant sheet is manufactured of biodegradable aromatic polyester.

6. The diaper as claimed in claim 5, wherein the biodegradable aromatic polyester is polylactic acid (PLA) derived from a plant source, which is compostable and biodegradable by at least 15 wt. % respectively.

7. The diaper as claimed in claim 1, wherein the top sheet is also manufactured from an aromatic polyester is polylactic acid (PLA) derived from a plant source, and biodegradable by at least 7 wt. % respectively.

8. The diaper as claimed in claim 1, wherein one or more of the biodegradable top sheet, top sheet, water resistant sheet, breathable bottom sheet are coated with a layer of cellulose fibre or are printed on using a plant based biofilm with water based inks.

9. The diaper as claimed in claim 1, having an inner core comprising first and second non-woven layers containing a fluff pulp material of a hydrophilic nature with a moisture content of less than 10% which is compostable and biodegradable by at least 27 wt. % respectively.

10. The diaper as claimed in claim 1, wherein the first non-woven layer, is arranged adjacent to the top sheet, comprises fibre particles with an average particle size that is smaller than the second-non-woven layer.

11. The diaper as claimed in claim 10, wherein the average particle size in the first non-woven layer is less than 3 mm.

12. The diaper as claimed in claim 1, wherein in combination with the non-woven layers there is a superabsorbent polymer (SAP) comprising mainly of sodium polyacrylate.

13. The diaper as claimed in claim 1, wherein a proportion or ratio of SAP to fluff pulp material mix is substantially between 1:0.5-2.5.

14. The diaper as claimed in claim 1, wherein a proportion or ratio of a first SAP to other Sandia SAP when combined is substantially 6:4.

15. The diaper as claimed in claim 1, further comprising superabsorbent fillers that are bio-based with content greater than 25%.

16. The diaper as claimed in claim 1, wherein the diaper comprises a core, side sheet, leak guard and/or double leak guards configured with 3D elastic that automatically position themselves such that there is no requirement to manually pull them out, thereby increasing an absorbency area, creating absorbency channels down the side of the core to prevent further leaks.

17. The diaper as claimed in claim 1, further comprising a leak guard, tabs, frontal tape portion, elastic waistband that comprise bio-based materials having a bio-based content that is greater than 25%.

18. The diaper as claimed in claim 1, wherein the diaper when sold comprises a 100% compostable outer bag such that a total nappy biodegradability package is greater than 90 wt. %.

19. The diaper of claim 1 further comprising a biodegradable water resistant outer layer that is liquid impervious, wherein the diaper materials are at least 90% biodegradable by volume (v/v %) and at least 90 wt. % compostable.
